(19) United States
(12) Patent Application Publication
(10) Pub. No.: US 2024/0404676 A1
(43) Pub. Date: Dec. 5, 2024

(54) METHOD AND SYSTEM FOR PROVIDING CONTEXTUAL BASED MEDICATION DOSAGE DETERMINATION

(71) Applicant: Abbott Diabetes Care Inc., Alameda, CA (US)

(72) Inventors: Kenneth J. DONIGER, Menlo Park, CA (US); Mark Kent SLOAN, Hayward, CA (US)

(21) Appl. No.: 18/736,306

(22) Filed: Jun. 6, 2024

Related U.S. Application Data
(63) Continuation of application No. 14/280,538, filed on May 16, 2014, now Pat. No. 12,040,067.
Application No. 14/280,538 is a continuation of application No. 12/032,617, filed on Feb. 15, 2008, now Pat. No. 8,732,188.
(60) Provisional application No. 60/890,492, filed on Feb. 18, 2007.

(51) Int. Cl. G16H 20/17; G06F 16/13; G06F 16/245; G06F 16/635; G06F 16/958; G06N 5/04; G06N 20/00

(52) U.S. Cl. CPC G16H 20/17; G06F 16/13; G06F 16/245; G06F 16/636; G06F 16/972; G06N 5/04; G06N 20/00

(57) ABSTRACT

Methods and devices for statistical determination of medication dosage level such as bolus amount based on contextual information are provided.

### RELATED APPLICATIONS

The present application is a continuation of U.S. patent application Ser. No. 14/280,538, filed May 16, 2014, which is a continuation of U.S. patent application Ser. No. 12/032,617, filed Feb. 15, 2008, now U.S. Pat. No. 8,732,188, which claims priority under § 35 U.S.C. 119(e) to U.S. provisional patent application No. 60/890,492 filed Feb. 18, 2007, entitled “Method And System For Providing Contextual Based Medication Dosage Determination”, and assigned to the Assignee of the present application, Abbott Diabetes Care Inc. of Alameda, California, the disclosures of each of which are incorporated herein by reference for all purposes.

### BACKGROUND

With increasing use of pump therapy for Type 1 diabetic patients, young and old alike, the importance of controlling the infusion device such as external infusion pumps is evident. Indeed, presently available external infusion devices typically include an input mechanism such as buttons through which the patient may program and control the infusion device. Such infusion devices also typically include a user interface such as a display which is configured to display information relevant to the patient's infusion progress, status of the various components of the infusion device, as well as other programmable information such as patient specific basal profiles.

The external infusion devices are typically connected to an infusion set which includes a cannula that is placed transcutaneously through the skin of the patient to infuse a select dosage of insulin based on the infusion device's programmed basal rates or any other infusion rates as prescribed by the patient's doctor. Generally, the patient is able to control the pump to administer additional doses of insulin during the course of wearing and operating the infusion device such as for, administering a carbohydrate bolus prior to a meal. Certain infusion devices include a food database that has associated therewith, an amount of carbohydrate, so that the patient may better estimate the level of insulin dosage needed for, for example, calculating a bolus amount.

However, in general, most estimation or calculation of a bolus amount for administration, or a determination of a suitable basal profile, for that matter, are educated estimates based on the patient's physiology as determined by the patient's doctor, or an estimate performed by the patient. Moreover, the infusion devices do not generally include enhancement features that would better assist the diabetic patients to control and/or manage the glucose levels.

In view of the foregoing, it would be desirable to have a method and device for providing insulin therapy determination and recommendation based on real time monitored analyte levels of the patient for proactive insulin therapy treatment to improve management of diabetes. In addition, it would be desirable to have a method and system for providing insulin therapy determination and recommendation based on contextual information including the user or patient's past dosage administration and associated patient physiological conditions.

### SUMMARY

In accordance with the various embodiments of the present disclosure, there are provided methods and systems for determining suitable medication dosage levels based on contextual information including prior dosage administration and/or physiological conditions.

### BRIEF DESCRIPTION OF THE DRAWINGS

FIG. 1 is a block diagram illustrating a therapy management system for practicing one embodiment of the present disclosure;

FIG. 2 is a block diagram of a fluid delivery device of FIG. 1 in one embodiment of the present disclosure;

FIG. 3 is a flow chart illustrating therapy management procedure based on real time monitored analyte levels in accordance with one embodiment of the present disclosure;

FIG. 4 is a flowchart illustrating analyte trend information updating procedure based on real time monitored analyte levels in accordance with one embodiment of the present disclosure;

FIG. 5 is a flowchart illustrating modified therapy management procedure based on real time monitored analyte levels in accordance with one embodiment of the present disclosure;

FIG. 6 is a flowchart illustrating contextual based dosage determination in accordance with one embodiment of the present disclosure; and

FIG. 7 is a flowchart illustrating contextual based dosage determination in accordance with one embodiment of the present disclosure.

### DETAILED DESCRIPTION

As described in detail below, in accordance with the various embodiments of the present disclosure, there are provided method and device for determining suitable medication dosage level based on contextual information including user's patient's past dosage administration levels.

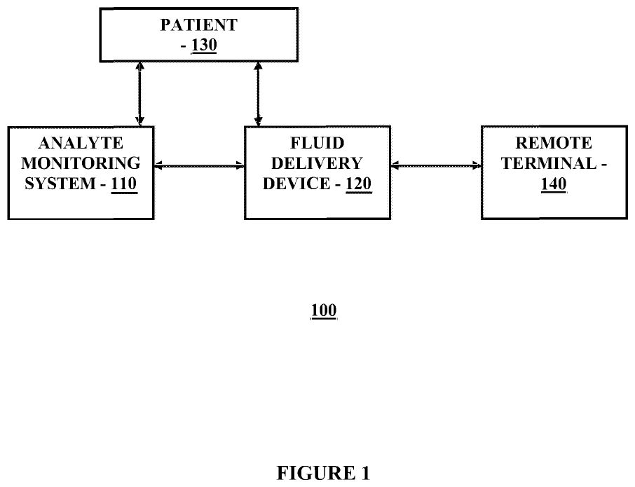

FIG. 1 is a block diagram illustrating an insulin therapy management system for practicing one embodiment of the present disclosure. Referring to FIG. 1, the therapy management system 100 includes an analyte monitoring system 110 operatively coupled to a fluid delivery device 120, which may be, in turn, operatively coupled to a remote terminal 140. As shown in the Figure, the analyte monitoring system 110 is, in one embodiment, coupled to the patient 130 so as to monitor or measure the analyte levels of the patient. Moreover, the fluid delivery device 120 is coupled to the patient using, for example, an infusion set and tubing connected to a cannula (not shown) that is placed transcutaneously through the skin of the patient so as to infuse medication such as, for example, insulin, to the patient.

Referring to FIG. 1, in one embodiment, the analyte monitoring system 110 may include one or more analyte sensors subcutaneously positioned such that at least a portion of the analyte sensors are maintained in fluid contact with the patient's analytes. The analyte sensors may include, but not limited to, short term subcutaneous analyte sensors or transdermal analyte sensors, for example, which are configured to detect analyte levels of a patient over a predetermined time period, and after which, a replacement of the sensors is necessary.

The one or more analyte sensors of the analyte monitoring system 110 is coupled to a respective one or more of a data transmitter unit which is configured to receive one or more signals from the respective analyte sensors corresponding to the detected analyte levels of the patient, and to transmit the information corresponding to the detected analyte levels to a receiver device, and/or fluid delivery device 120. That is, over a communication link, the transmitter units may be configured to transmit data associated with the detected analyte levels periodically, and/or intermittently and repeatedly to one or more other devices such as the insulin delivery device and/or the remote terminal 140 for further data processing and analysis.

The transmitter units of the analyte monitoring system 110 may be in one embodiment configured to transmit the analyte related data substantially in real time to the fluid delivery device 120 and/or the remote terminal 140 after receiving it from the corresponding analyte sensors such that the analyte level such as glucose level of the patient 130 may be monitored in real time. In one aspect, the analyte levels of the patient may be obtained using one or more of a discrete blood glucose testing device such as a blood glucose meter, or a continuous analyte monitoring system such as a continuous glucose monitoring system.

Analytes that may be monitored, determined or detected by the analyte monitoring system 110 include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined.

Moreover, within the scope of the present disclosure, the transmitter units of the analyte monitoring system 110 may be configured to directly communicate with one or more of the remote terminal 140 or the fluid delivery device 120. Furthermore, within the scope of the present disclosure, additional devices may be provided for communication in the analyte monitoring system 110 including additional receiver/data processing units, remote terminals (such as a physician's terminal and/or a bedside terminal in a hospital environment, for example. In addition, within the scope of the present disclosure, one or more of the analyte monitoring system 110, the fluid delivery device 120 and the remote terminal 140 may be configured to communicate over a wireless data communication link such as, but not limited to, RF communication link, Bluetooth● communication link, infrared communication link, or any other type of suitable wireless communication connection between two or more electronic devices, which may further be uni-directional or bi-directional communication between the two or more devices. Alternatively, the data communication link may include wired cable connection such as, for example, but not limited to, RS232 connection, USB connection, or serial cable connection.

Referring back to FIG. 1, in one embodiment, the analyte monitoring system 100 includes a strip port configured to receive a test strip for capillary blood glucose testing. In one aspect, the glucose level measured using the test strip may in addition, be configured to provide periodic calibration of the analyte sensors of the analyte monitoring system 110 to assure and improve the accuracy of the analyte levels detected by the analyte sensors.

Exemplary analyte systems that may be employed are described in, for example, U.S. Pat. Nos. 6,134,461, 6,175,752, 6,121,611, 6,560,471, 6,746,582, and elsewhere.

Referring again to FIG. 1, the fluid delivery device 120 may include in one embodiment, but not limited to, an external infusion device such as an external insulin infusion pump, an implantable pump, a pen-type insulin injector device, an on-body patch pump, an inhalable infusion device for nasal insulin delivery, or any other type of suitable delivery system. In addition, the remote terminal 140 in one embodiment may include for example, a desktop computer terminal, a data communication enabled kiosk, a laptop computer, a handheld computing device such as a personal digital assistant (PDAs), or a data communication enabled mobile telephone.

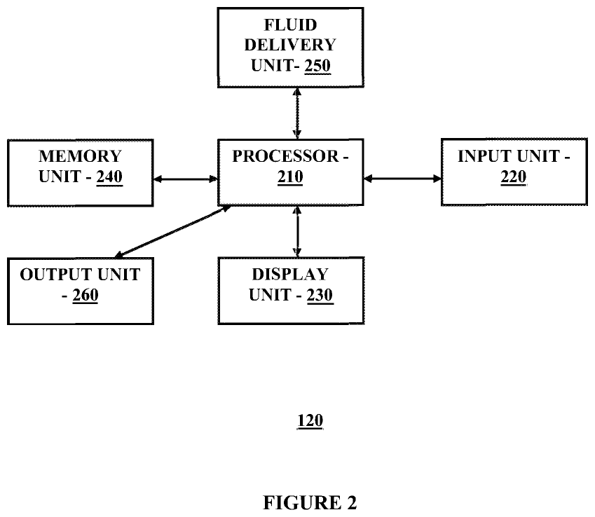

FIG. 2 is a block diagram of an insulin delivery device of FIG. 1 in one embodiment of the present disclosure. Referring to FIG. 2, the fluid delivery device 120 in one embodiment includes a processor 210 operatively coupled to a memory unit 240, an input unit 220, a display unit 230, an output unit 260, and a fluid delivery unit 250. In one embodiment, the processor 210 includes a microprocessor that is configured to and capable of controlling the functions of the fluid delivery device 120 by controlling and/or accessing each of the various components of the fluid delivery device 120. In one embodiment, multiple processors may be provided as a safety measure and to provide redundancy in case of a single processor failure. Moreover, processing capabilities may be shared between multiple processor units within the insulin delivery device 120 such that pump functions and/or control may be performed faster and more accurately.

Referring back to FIG. 2, the input unit 220 operatively coupled to the processor 210 may include a jog dial, key pad buttons, a touch pad screen, or any other suitable input mechanism for providing input commands to the fluid delivery device 120. More specifically, in case of a jog dial input device, or a touch pad screen, for example, the patient or user of the fluid delivery device 120 will manipulate the respective jog dial or touch pad in conjunction with the display unit 230 which performs as both a data input and output units. The display unit 230 may include a touch sensitive screen, an LCD screen, or any other types of suitable display unit for the fluid delivery device 120 that is configured to display alphanumeric data as well as pictorial information such as icons associated with one or more predefined states of the fluid delivery device 120, or graphical representation of data such as trend charts and graphs associated with the insulin infusion rates, trend data of monitored glucose levels over a period of time, or textual notification to the patients.

Referring to FIG. 2, the output unit 260 operatively coupled to the processor 210 may include an audible alarm including one or more tones and/or preprogrammed or programmable tunes or audio clips, or vibratory alert features having one or more pre-programmed or programmable vibratory alert levels. In one embodiment, the vibratory alert may also assist in priming the infusion tubing to minimize the potential for air or other undesirable material in the infusion tubing. Also shown in FIG. 2 is the fluid delivery unit 250 which is operatively coupled to the processor 210 and configured to deliver the insulin doses or amounts to the patient from the insulin reservoir or any other types of suitable containment for insulin to be delivered (not shown) in the fluid delivery device 120 via an infusion set coupled to a subcutaneously positioned cannula under the skin of the patient.

Referring yet again to FIG. 2, the memory unit 240 may include one or more of a random access memory (RAM), read only memory (ROM), or any other types of data storage units that are configured to store data as well as program instructions for access by the processor 210 and execution to control the fluid delivery device 120 and/or to perform data processing based on data received from the analyte monitoring system 110, the remote terminal 140, the patient 130 or any other data input source.

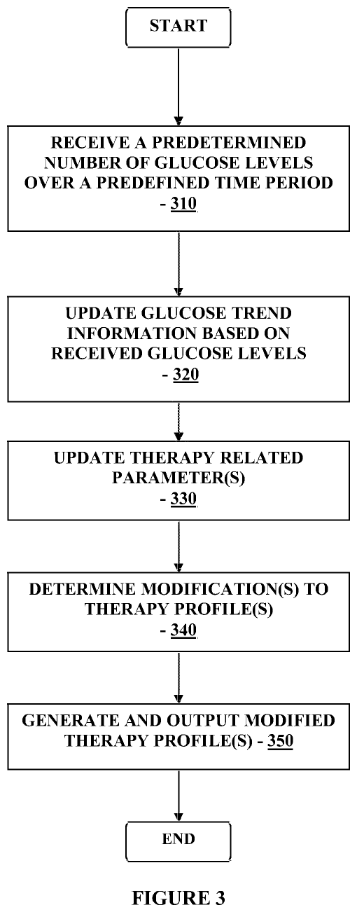

FIG. 3 is a flow chart illustrating insulin therapy management procedure based on real time monitored analyte levels in accordance with one embodiment of the present disclosure. Referring to FIG. 3, in one embodiment of the present disclosure, a predetermined number of consecutive glucose levels are received or detected over a predetermined or defined time period. For example, in one embodiment, referring to FIG. 1, the monitored glucose level of a patient is substantially continuously received or detected substantially in real time for a predetermined time period. In one embodiment, the predefined time period may include one or more time periods, the data within which may provide a therapeutically meaningful basis for associated data analysis.

That is, the predefined time period of the real time monitored glucose data in one embodiment may include one or more time periods sufficient to provide glucose trend information or sufficient to provide analysis of glucose levels to adjust insulin therapy on an on-going, and substantially real time basis. For example, the predefined time period in one embodiment may include one or more of a 15 minute time period, a 30 minute time period, a 45 minute time period, a one hour time period, a two hour time period and a 6 hour time period. While exemplary predefined time periods are provided herein, within the scope of the present disclosure, any suitable predefined time period may be employed as may be sufficient to be used for glucose trend determination and/or therapy related determinations (such as, for example, modification of existing basal profiles, calculation of temporary basal profile, or determination of a bolus amount).

Referring back to FIG. 3, the consecutive glucose levels received over the predefined time period in one embodiment may not be entirely consecutive due to, for example, data transmission errors and/or one or more of potential failure modes associated with data transmission or processing. As such, in one embodiment of the present disclosure, there is provided a predetermined margin of error for the received real time glucose data such that, a given number of data points associated with glucose levels which are erroneous or alternatively, not received from the glucose sensor, may be ignored or discarded.

Referring back to FIG. 3, upon receiving the predetermined number of glucose levels over a predefined time period, the glucose trend information based on the received glucose levels are updated. For example, in one embodiment, the glucose trend information estimating the rate of change of the glucose levels may be determined, and based upon which the projected level of glucose may be calculated. Indeed, in one embodiment, the glucose trend information may be configured to provide extrapolated glucose level information associated with the glucose level movement based on the real time glucose data received from the glucose sensor. That is, in one embodiment, the real time glucose levels monitored are used to determine the rate at which the glucose levels are either increasing or decreasing (or remaining substantially stable at a given level). Based on such information and over a predetermined time period, a glucose projected information may be determined.

Referring again to FIG. 3, the therapy related parameters associated with the monitored real time glucose levels are updated. That is, in one embodiment, one or more insulin therapy related parameters of an insulin pump such as, but not limited to, insulin on board information associated with the fluid delivery device 120 (FIG. 1), insulin sensitivity level of the patient 130 (FIG. 1), insulin to carbohydrate ratio, and insulin absorption rate. Thereafter, in one embodiment, one or more modifications to the current therapy profile are determined. That is, in one embodiment of the present disclosure, one or more current basal profiles, calculated bolus levels, temporary basal profiles, and/or any other suitable pre-programmed insulin delivery profiles stored in the fluid delivery device 120 (FIG. 1) for example, are retrieved and analyzed based on one or more of the received real time glucose levels, the updated glucose trend information, and the updated therapy related parameters.

Referring back to FIG. 3, after determining one or more modifications to the therapy profiles, the modified one or more therapy profiles are generated and output to the patient 130 (FIG. 1) so that the patient 130 may select, store and/or ignore the one or more modified therapy profiles based on one or more of the monitored real time glucose values, updated glucose trend information, and updated therapy related parameters.

For example, in one embodiment, the patient 130 may be provided with a recommended temporary basal profile based on the monitored real time glucose levels over a predetermined time period as well as the current basal profile which is executed by the fluid delivery device 120 (FIG. 1) to deliver a predetermined level of insulin to the patient 130 (FIG. 1). Alternatively, the patient 130 in a further embodiment may be provided with one or more additional recommended actions for selection as the patient sees suitable to enhance the insulin therapy based on the real time monitored glucose levels. For example, the patient may be provided with a recommended correction bolus level based on the real time monitored glucose levels and the current basal profile in conjunction with, for example, the patient's insulin sensitivity and/or insulin on board information.

In this manner, in one embodiment of the present disclosure, based on real time monitored glucose levels, the patient may be provided with on-going, real time insulin therapy options and modifications to the pre-programmed insulin delivery basal profiles so as to improve upon the initially programmed therapy profiles based on the monitored real time glucose data.

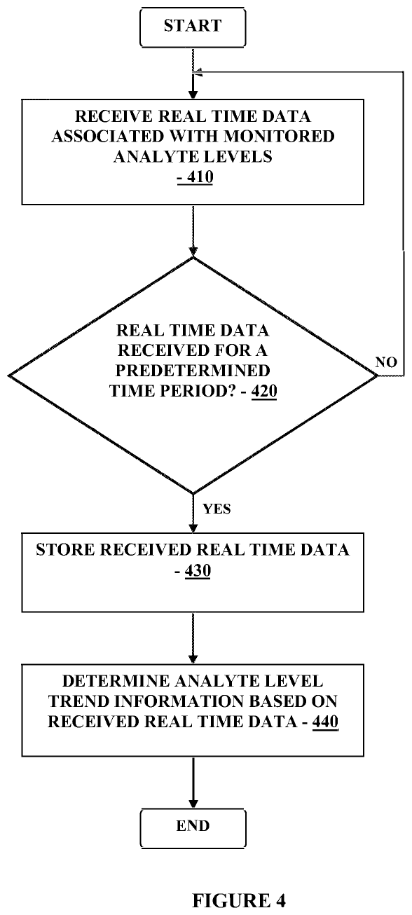

FIG. 4 is a flowchart illustrating analyte trend information updating procedure based on real time monitored analyte levels in accordance with one embodiment of the present disclosure. Referring to FIG. 4, in one embodiment, real time data associated with monitored analyte levels is received at step 410. Thereafter it is determined whether the real time data has been received for a predetermined time period at step 420. If it is determined that the real time data has not been received for at least the predetermined time period, then the routine continues to receive the real time data associated with the monitored analyte levels such as glucose levels.

On the other hand, referring back to FIG. 4, if it is determined that the real time data associated with the monitored analyte levels have been received for the predetermined time period (for example, as described above in conjunction with FIG. 3), then, the received real time data associated with the monitored analyte levels are stored at step 430. Thereafter, analyte level trend information is determined based on the received real time data at step 440 associated with the monitored analyte levels.

For example, in one embodiment, the real time data associated with the monitored analyte levels is analyzed and an extrapolation of the data based on the rate of change of the monitored analyte levels is determined. That is, the real time data associated with the monitored analyte levels is used to determined the rate at which the monitored analyte level changed over the predetermined time period, and accordingly, a trend information is determined based on, for example, the determined rate at which the monitored analyte level changed over the predetermined time period.

In a further embodiment, the trend information based on the real time data associated with the monitored analyte levels may be dynamically modified and continuously updated based on the received real time data associated with the monitored analyte levels for one or more predetermined time periods. As such, in one embodiment, the trend information may be configured to dynamically change and be updated continuously based on the received real time data associated with the monitored analyte levels.

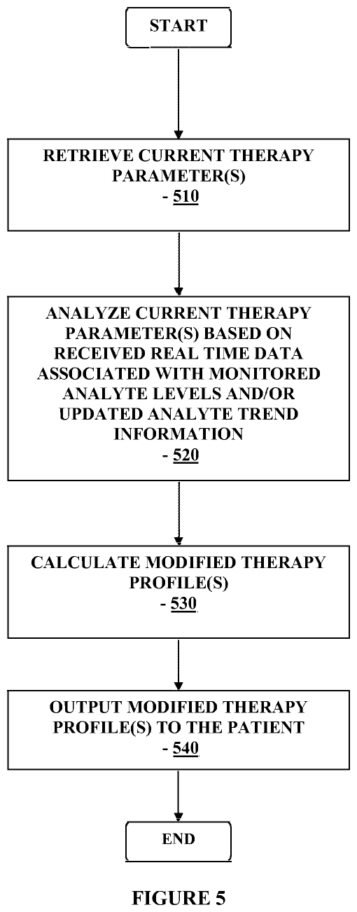

FIG. 5 is a flowchart illustrating modified therapy management procedures based on real time monitored analyte levels in accordance with one embodiment of the present disclosure. Referring to FIG. 5, in one embodiment, the current therapy parameters are retrieved at step 510 and the retrieved current therapy parameters are analyzed based on the received real time data associated with the monitored analyte levels and/or updated analyte trend information at step 520. For example, one or more preprogrammed basal profiles, correction bolus, carbohydrate bolus, temporary basal and associated parameters are retrieved and analyzed based on, for example, the received real time data associated with the monitored analyte levels and/or updated analyte trend information, and further, factoring in the insulin sensitivity of the patient as well as insulin on board information.

Referring to FIG. 5, based upon the analysis of the current therapy parameters, one or more modified therapy profiles are calculated at step 530. That is, based upon the real time glucose levels monitored by the analyte monitoring system 110 (FIG. 1), a modification or adjustment to the pre-programmed basal profiles of the fluid delivery device 120 (FIG. 1) may be determined, and the modified therapy profile is output to the patient 130 (FIG. 1) at step 540. That is, the modification or adjustment to the pre-programmed basal profiles may be provided to the patient for review and/or execution to implement the recommended modification or adjustment to the pre-programmed basal profiles.

In this manner, the patient may be provided with one or more adjustments to the existing or current basal profiles or any other pre-programmed therapy profiles based on continuously monitored physiological levels of the patient such as analyte levels of the patient. Indeed, in one embodiment of the present disclosure, using continuously monitored glucose levels of the patient, modification or adjustment to the pre-programmed basal profiles may be calculated and provided to the patient for review and implementation as desired by the patient. In this manner, for example, a diabetic patient may improve the insulin therapy management and control.

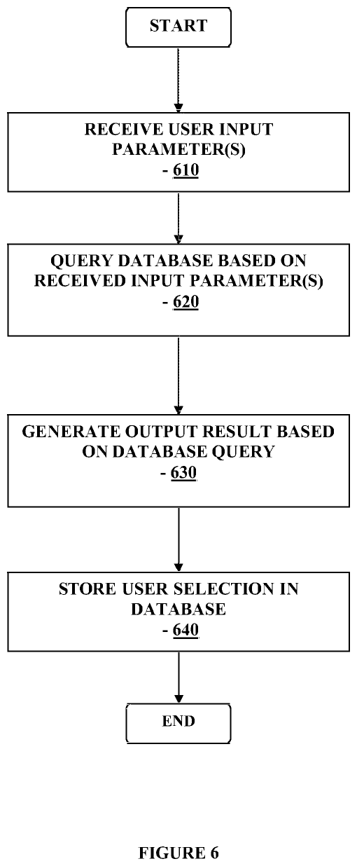

FIG. 6 is a flowchart illustrating contextual based dosage determination in accordance with one embodiment of the present disclosure. Referring to the Figure, one or more user input parameter is received at step 610 such as, for example, the amount of carbohydrate to ingest, type of exercise to perform, current time of day information, or any other appropriate information that may potentially impact the determination of the suitable medication level. Based on the one or more user input parameters, one or more database is queried at step 620. In one embodiment, the database may be provided in the analyte monitoring system 110. Alternatively or in addition, the one or more database may be provided in the fluid delivery device 120 and/or remote terminal 140.

Referring back to FIG. 6, the database query in one embodiment may be configured to search or query for medication dosage levels that are associated with similar parameters as the received one or more user input parameters. Thereafter, the queried result is generated at step 630 and provided to the user which may be acted upon by the user, for example, to administer the medication dosage level based on the queried result. The user selection of the administered medication dosage level is stored in the database at step 640 with the associated one or more user input parameters as well as the time and date information of when the user has administered the medication dosage level.

In this manner, in one embodiment, insulin dosages and associated contextual information (e.g., user input parameters) may be stored and tracked in one or more databases. For example, a bolus amount for a diabetic patient may be determined in the manner described above using historical information without performing a mathematical calculation which takes into account variables, such as sensitivity factors, that vary with time and/or user's physiological conditions, and which may need to be estimated.

In particular, in one embodiment of the present disclosure, insulin dependent users may determine their appropriate insulin dosages by, for example, using historical dosage information as well as associated physiological condition information. For example, the historical data may be stored in one or more databases to allow search or query based on one or more parameters such as the user's physiological condition and other contextual information associated with each prior bolus dosage calculated and administered. In this manner, the user may be advised on the proper amount of insulin under the particular circumstances, the user may be provided with descriptive statistical information of insulin dosages under the various conditions, and the overall system may be configured to learn and customize the dosage determination for the particular user over an extended time period.

For example, in one aspect, contextual information may be stored with the insulin bolus value. The contextual data in one aspect may include one or more of blood glucose concentration, basal rate, type of insulin, exercise information, meal information, carbohydrate content estimate, insulin on board information, and any other parameters that may be used to determine the suitable or appropriate medication dosage level. Some or all of the contextual information may be provided by the user or may be received from another device or devices in the overall therapy management system such as receiving the basal rate information from the fluid delivery device 120 (FIG. 1), or receiving the blood glucose concentration from the analyte monitoring system 110 (FIG. 1).

By way of an example, a contextually determined medication dosage level in one embodiment may be provided to the user along with a suitable or appropriate notification or message to the user that after a predetermined time period since the prior administration of the medication dosage level, the blood glucose level was still above a target level. That is, the queried result providing the suitable medication dosage level based on user input or other input parameters may be accompanied by other relevant physiological condition information associated with the administration of the prior medication dosage administration. In this manner, when the user is provided with the contextually determined medication dosage level, the user is further provided with information associated with the effects of the determined medication dosage level to the user's physiological condition (for example, one hour after the administration of the particular medication dosage level determined, the user's blood glucose level changed by a given amount). Accordingly, the user may be better able to adjust or modify, as desired or needed, the contextually determined medication dosage level to the current physiological conditions.

In this manner, in one embodiment, to determine and provide the user with proper medication dosage levels, the present or current context including the patient's current physiological condition (such as current blood glucose level, current glucose trend information, insulin on board information, the current basal profile, and so on) is considered and the database is queried for one or more medication dosage levels which correlate (for example, within a predetermined range of closeness or similarity) to the one or more current contextual information associated with the user's physiological condition, among others.

Accordingly, in one embodiment, statistical determination of the suitable medication dosage based on contextual information may be determined using, one or more of mean dosage determination, using a standard deviation or other appropriate statistical analysis of the contextual information for medication dosages which the user has administered in the past. Further, in one aspect, in the case where no close match is found in the contextual query for the desired medication dosage level, the medication dosage level with the most similar contextual information may be used to interpolate an estimated medication dosage level.

In still another aspect, the database query may be configured to provide time based weighing of prior medication dosage level determinations such that, for example, more recent dosage level determination in which similar contextual information may be weighed heavier than aged dosage level determination under similar conditions. For example, older or more aged bolus amounts determined may be weighed less heavily than the more recent bolus amounts. Also, over an extended period of time, in one aspect, the older or aged bolus amounts may be aged out or weighed with a value parameter that minimally impacts the current contextual based bolus determination. In this manner, in one aspect, a highly personalized and individualistic profile for medication dosage determination may be developed and stored in the database with the corresponding contextual information associated therewith.

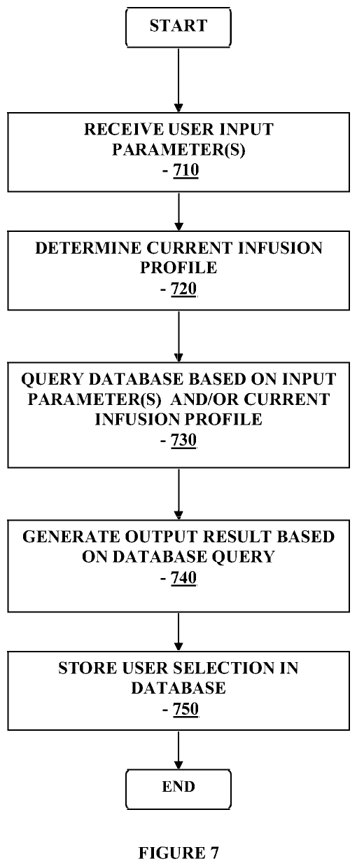

FIG. 7 is a flowchart illustrating contextual based dosage determination in accordance with one embodiment. Referring to FIG. 7, in one aspect, when the user input parameters are received at step 710, the current infusion profile of the user's insulin pump is determined at step 720. Thereafter, the database is queried based on the input parameters and the current infusion profile at step 730, and which results in one or more contextually determined bolus amount associated with the input parameters and the current infusion profile at step 740 that is provided to the user. The determined bolus amount is then stored in the database at step 750 with the associated input parameters and the current infusion profile and any other contextual information associated with the determined bolus amount.

In this manner, in one aspect, in addition to the user provided input parameters, other relevant contextual information may be retrieved (for example, the current infusion profile such as basal rate from the insulin pump, the current blood glucose level and/or glucose trend information from the analyte monitoring system, and the like) prior to the database query to determine the suitable bolus amount.

As discussed above, optionally, the contextual information including the user input parameters and other relevant information may be queried to determine the suitable medication dosage level based on one or more statistical analysis such as, for example, but not limited to, descriptive statistics with the use of numerical descriptors such as mean and standard deviation, or inferential statistics including, for example, estimation or forecasting, correlation of parameters, modeling of relationships between parameters (for example, regression), as well as other modeling approaches such as time series analysis (for example, autoregressive modeling, integrated modeling and moving average modeling), data mining, and probability.

By way of a further non-limiting example, when a diabetic patient plans to ingest insulin of a particular type, the patient enters contextual information such as that the patient has moderately exercised and is planning to consume a meal with a predetermined estimated carbohydrate content. The database in one embodiment may be queried for insulin dosages determined under similar circumstances in the past for the patient, and further, statistical information associated with the determined insulin dosage is provided to the user. In one aspect, the displayed statistical information associated with the determined insulin dosage may include, for example, an average amount of insulin dosage, a standard deviation or a median amount and the 25^{th} and the 75^{th} percentile values of the determined insulin dosage.

The patient may consider the displayed statistical information associated with the determined insulin dosage, and determine the most suitable or desired insulin amount based on the information received. When the patient programs the insulin pump to administer the desired insulin amount (or otherwise administer the desired insulin amount using other medication administration procedures such as injection (using a pen-type injection device or a syringe), intaking inhalable or ingestible insulin, and the like), the administered dosage level is stored in the database along with the associated contextual information and parameters.

In this manner, the database for use in the contextual based query may be continuously updated with each administration of the insulin dosage such that, each subsequent determination of appropriate insulin dosage level may be determined with more accuracy and is further customized to the physiological profile of the particular patient. Additionally, the database queried may be used for other purposes, such as, for example, but not limited to, tracking medication information, providing electronic history of the patient related medical information, and the like. Further, while the above example is provided in the context of determining an insulin level determination, within the scope of the present disclosure, other medication dosage may be determined based on the contextual based database query approaches described herein.

In a further aspect, the contextual based medication dosage query and determination may be used in conjunction with the standard or available medication dosage determination (for example, standard bolus calculation algorithms) as a supplement to provide additional information or provide a double checking ability to insure that the estimated or calculated bolus or medication dosage level is appropriate for the particular patient under the physiological condition at the time of the dosage level determination.

In still a further aspect, user or patient feedback on current or prior medication dosage levels may be used in conjunction with the contextual based medication dosage query and determination to improve the user or patient's therapy management.

Within the scope of the present disclosure, the processes and routines described in conjunction with FIGS. 3-7 may be performed by the analyte monitoring system 110 (FIG. 1) and/or the fluid delivery device 120 (FIG. 1). Furthermore, the output of information associated with the context based database query for medication dosage determination may be displayed on a display unit of the receiver of the analyte monitoring system 110 (FIG. 1), or the infusion device display of the fluid delivery device 120 (FIG. 1), the display unit of the remote terminal 140 (FIG. 1), or any other suitable output device that is configured to receive the results of the database query associated with the medication dosage level determination. Alternatively, one or more such information may be output to the patient audibly as sound signal output.

In this manner, there are provided methods and system for receiving one or more parameters associated with a user physiological condition, querying a database based on the one or more parameters associated with the user physiological condition, generating a medication dosage amount based on the database query, and outputting the medication dosage amount to the user.

Optionally, statistical analysis may be performed based on the database query and factored into generating the medication dosage amount for the user.

In other aspects, there are provided methods and systems for providing information associated with the direction and rate of change of analyte (e.g., glucose) levels changes for determination of, for example, bolus or basal rate change recommendations, for comparing expected glucose level changes to actual real time glucose level changes to update, for example, insulin sensitivity factor in an ongoing basis, and for automatically confirming the monitored glucose values within a preset time period (e.g., 30 minutes) after insulin therapy initiation to determine whether the initiated therapy is having the intended therapeutic effect.

Indeed, in accordance with the various embodiments of the present disclosure, the use of glucose trend information in insulin delivery rate determinations provides for a more accurate insulin dosing and may lead to a decrease in hypoglycemic events and improved HbA1Cs.

A method in one embodiment includes receiving one or more parameters associated with a physiological condition, querying a database based on the one or more parameters associated with the physiological condition, generating a medication level information based on the database query, and outputting the medication level information.

The database may include a plurality of medication level information and one or more associated parameters for each of the plurality of the medication level information.

The one or more of the plurality of the medication level information stored in the database may include administered medication dosage information, where the administered medication dosage information may include one or more of a past correction bolus amount, a past carbohydrate bolus amount, a past basal profile, or one or more combinations thereof.

Further, querying the database may include performing statistical analysis based on the received one or more parameters, where the statistical analysis may include one or more of mean deviation analysis, standard deviation analysis, estimation analysis, forecasting analysis, correlation of the one or more parameters, modeling of one or more relationships among the one or more parameters, regression analysis, time series analysis, autoregressive modeling, integrated modeling, moving average modeling, data mining, or probability analysis.

The generated medication level information may include one or more of a bolus amount or a basal profile modification for administration to a user.

The method in a further embodiment may include receiving a command associated with the outputted mediation level information.

In another aspect, the method may include executing a therapy related operation based on the outputted medication level information, where the therapy related operation may include generating a command to infuse medication based on the outputted medication level information.

In still another aspect, the method may also include receiving a command confirming administration of a medication amount associated with the outputted medication level information, and storing the outputted medication level information.

The medication level information may be associated with insulin. For example, the medication level information may include a bolus dosage amount, a modification to a predetermined basal profile or a new basal profile for infusing insulin.

The one or more parameters may include one or more of a current glucose level, an insulin sensitivity, a target glucose level, past glucose level, glucose trend information, an amount of carbohydrate to be ingested, insulin on board information, exercise information, time of day information, or one or more combinations thereof.

An apparatus in another embodiment may include one or more processing units, and a memory for storing instructions which, when executed by the one or more processing units, causes the one or more processing units to receive one or more parameters associated with a physiological condition, query the memory based on the one or more parameters associated with the physiological condition, generate a medication level information based on the database query, and output the medication level information.

The memory may include a plurality of medication level information and one or more associated parameters for each of the plurality of the medication level information. In one aspect, the memory may include one or more memory devices including, random access memory (RAM), read-only memory (ROM), electrically erasable programmable read only memory (EEPROM), erasable programmable read-only memory (EPROM), or combinations thereof.

The one or more of the plurality of the medication level information stored in the memory may include administered medication dosage information, where the administered medication dosage information may include one or more of a past correction bolus amount, a past carbohydrate bolus amount, a past basal profile, or one or more combinations thereof.

The memory for storing instructions which, when executed by the one or more processing units, may cause the one or more processing units to perform statistical analysis based on the received one or more parameters, where the statistical analysis may include one or more of mean deviation analysis, standard deviation analysis, estimation analysis, forecasting analysis, correlation of the one or more parameters, modeling of one or more relationships among the one or more parameters, regression analysis, time series analysis, autoregressive modeling, integrated modeling, moving average modeling, data mining, or probability analysis.

The generated medication level information may include one or more of a bolus amount or a basal profile modification for administration to a user.

The memory for storing instructions which, when executed by the one or more processing units, may in another aspect, cause the one or more processing units to receive a command associated with the outputted mediation level information.

The memory for storing instructions which, when executed by the one or more processing units, may cause the one or more processing units to execute a therapy related operation based on the outputted medication level information.

The memory for storing instructions which, when executed by the one or more processing units, may cause the one or more processing units to generate a command to infuse medication based on the outputted medication level information.

The memory for storing instructions which, when executed by the one or more processing units, may cause the one or more processing units to receive a command confirming administration of a medication amount associated with the outputted medication level information, and to store the outputted medication level information.

The medication level information is associated with insulin.

The one or more parameters includes one or more of a current glucose level, an insulin sensitivity, a target glucose level, past glucose level, glucose trend information, an amount of carbohydrate to be ingested, insulin on board information, exercise information, time of day information, or one or more combinations thereof.

In one aspect, the one or more processing units, and the memory are operatively coupled to a medical device, where the medical device may include a blood glucose meter, an analyte monitoring device, or an infusion device. Further, the at least two of the blood glucose meter, the analyte monitoring device or the infusion device may be integrated in a single housing.

A computer program product for enabling one or more processors to perform a database query in a further aspect includes a computer readable medium, and software instructions on the computer readable medium, for enabling the one or more processors to perform predetermined operations comprising receiving one or more parameters associated with a physiological condition, querying a database based on the one or more parameters associated with the physiological condition, generating a medication level information based on the database query, and outputting the medication level information.

Indeed, in one aspect, the medication level information determination and data processings related to the determination may be integrated in the analyte monitoring system 110, the fluid delivery device 120, or the remote terminal 140 (FIG. 1). Alternatively, in networked environment, the memory or database may be located separately from the one or more processing units such that the device incorporating the one or more processing units (such as the fluid delivery device 120 or the analyte monitoring system 110) may be configured to access the information stored in the database or memory in the remote terminal 140 (FIG. 1) over a data network optionally using data encryption, to retrieve and/or store data therein.

A method in another embodiment may include receiving data associated with monitored analyte related levels for a predetermined time period substantially in real time, retrieving one or more therapy profiles associated with the monitored analyte related levels, generating one or more modifications to the retrieved one or more therapy profiles based on the data associated with the monitored analyte related levels.

The method may further include displaying the generated one or more modifications to the retrieved one or more therapy profiles.

In one aspect, the generated one or more modifications to the retrieved one or more therapy profiles may be displayed as one or more of an alphanumeric output display, a graphical output display, an icon display, a video output display, a color display or an illumination display.

In a further aspect, the predetermined time period may include one of a time period between 15 minutes and six hours.

The one or more therapy profiles in yet another aspect may include a basal profile, a correction bolus, a temporary basal profile, an insulin sensitivity, an insulin on board level, and an insulin absorption rate.

In still another aspect, retrieving the one or more therapy profiles associated with the monitored analyte related levels may include retrieving a current analyte rate of change information.

In yet still another aspect, generating the one or more modifications to the retrieved one or more therapy profiles may include determining a modified analyte rate of change information based on the received data associated with monitored analyte related levels.

Moreover, the method may further include generating an output alert based on the modified analyte rate of change information.

Still, the method may also include determining an analyte level projection information based on the modified analyte rate of change information.

A system for providing diabetes management in accordance with another embodiment of the present disclosure includes an interface unit, one or more processors coupled to the interface unit, a memory for storing instructions which, when executed by the one or more processors, causes the one or more processors to receive data associated with monitored analyte related levels for a predetermined time period substantially in real time, retrieve one or more therapy profiles associated with the monitored analyte related levels, and generate one or more modifications to the retrieved one or more therapy profiles based on the data associated with the monitored analyte related levels.

The interface unit may include an input unit and an output unit, the input unit configured to receive the one or more analyte related data, and the output unit configured to output the one or more of the generated modifications to the retrieved one or more therapy profiles.

The interface unit and the one or more processors in a further embodiment may be operatively coupled to one or more of a housing of an infusion device or a housing of an analyte monitoring system.

The infusion device may include one of an external insulin pump, an implantable insulin pump, an on-body patch pump, a pen-type injection device, an inhalable insulin delivery system, and a transdermal insulin delivery system.

The memory in a further aspect may be configured for storing instructions which, when executed by the one or more processors, causes the one or more processors to display the generated one or more modifications to the retrieved one or more therapy profiles.

Further, the memory may be configured for storing instructions which, when executed by the one or more processors, causes the one or more processors to display the generated one or more modifications to the retrieved one or more therapy profiles as one or more of an alphanumeric output display, a graphical output display, an icon display, a video output display, a color display or an illumination display.

In one aspect, the predetermined time period may include one of a time period between 15 minutes and six hours.

The one or more therapy profiles may include a basal profile, a correction bolus, a temporary basal profile, an insulin sensitivity, an insulin on board level, and an insulin absorption rate.

In another aspect, the memory may be further configured for storing instructions which, when executed by the one or more processors, causes the one or more processors to retrieve a current analyte rate of change information.

In still another aspect, the memory may be further configured for storing instructions which, when executed by the one or more processors, causes the one or more processors to determine a modified analyte rate of change information based on the received data associated with monitored analyte related levels.

Additionally, in yet still another aspect, the memory may be further configured for storing instructions which, when executed by the one or more processors, causes the one or more processors to generate an output alert based on the modified analyte rate of change information.

Further, the memory may be further configured for storing instructions which, when executed by the one or more processors, causes the one or more processors to determine an analyte level projection information based on the modified analyte rate of change information.

A system for providing diabetes management in accordance with yet another embodiment of the present disclosure includes an analyte monitoring system configured to monitor analyte related levels of a patient substantially in real time, a medication delivery unit operatively for wirelessly receiving data associated with the monitored analyte level of the patient substantially in real time from the analyte monitoring system, a data processing unit operatively coupled to the one or more of the analyte monitoring system or the medication delivery unit, the data processing unit configured to retrieve one or more therapy profiles associated with the monitored analyte related levels, and generate one or more modifications to the retrieved one or more therapy profiles based on the data associated with the monitored analyte related levels.

In one aspect, the analyte monitoring system may be configured to wirelessly communicate with the medication delivery unit over a radio frequency (RF) communication link, a Bluetooth● communication link, an Infrared communication link, or a local area network (LAN).

The various processes described above including the processes performed by the processor 210 in the software application execution environment in the fluid delivery device 120 as well as any other suitable or similar processing units embodied in the analyte monitoring system 110 and the remote terminal 140, including the processes and routines described in conjunction with FIGS. 3-7, may be embodied as computer programs developed using an object oriented language that allows the modeling of complex systems with modular objects to create abstractions that are representative of real world, physical objects and their interrelationships. The software required to carry out the inventive process, which may be stored in the memory unit 240 (or similar storage devices in the analyte monitoring system 110 and the remote terminal 140) of the processor 210, may be developed by a person of ordinary skill in the art and may include one or more computer program products.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present disclosure and that structures and methods within the scope of these claims and their equivalents be covered thereby.

1-20. (canceled)

21. A method for operating an insulin pump, comprising:
monitoring analyte levels by an analyte monitor over a predetermined period of time in which the insulin pump is configured to administer insulin according to a first rate specified by an insulin therapy profile, wherein the analyte monitor comprises a subcutaneous analyte sensor and a transmitter;
retrieving the insulin therapy profile and a predetermined parameter, wherein the predetermined parameter comprises insulin delivery information during the predetermined period of time;
determining a modification based on the insulin therapy profile and at least one of the retrieved insulin delivery information and the monitored analyte levels; and
adjusting the insulin therapy profile based on the determined modification to generate an adjusted insulin therapy profile, wherein the adjusted insulin delivery therapy comprises a second rate for administering the insulin.

22. The method of claim 21, wherein the insulin therapy profile comprises at least one of a current basal profile, a temporary basal profile, or a pre-programmed insulin delivery profile.

23. The method of claim 21, further comprising:
outputting the adjusted insulin therapy profile to a user device; and
receiving, from the user device, an instruction to process the adjusted insulin therapy profile.

24. The method of claim 23, wherein the instruction to process the adjusted insulin therapy profile comprises at least one of selecting the adjusted insulin therapy profile, storing the adjusted insulin therapy profile, or ignoring the adjusted insulin therapy profile.

25. The method of claim 24, wherein when the instruction to process the adjusted insulin therapy profile comprises selecting the adjusted insulin therapy profile, the instruction is configured to cause the insulin pump to operate based on the second rate for administering the insulin.

26. The method of claim 21, wherein the second rate for administering the insulin comprises a temporary basal rate.

27. The method of claim 21, wherein the adjusted insulin therapy profile further comprises a new bolus amount determined based on a user input parameter.

28. The method of claim 27, wherein the user input parameter comprises at least one of an amount of carbohydrate to ingest, a type of exercise to perform, and a current time of day information.

29. The method of claim 27, further comprising storing the new bolus amount in a database, and wherein the database further includes a plurality of previously determined medication dosage levels and one or more associated parameters for each of the plurality of the previously determined medication dosage levels.

30. The method of claim 29, wherein one or more of the plurality of the previously determined medication dosage levels stored in the database includes administered medication dosage information.

31. The method of claim 29, further including performing statistical analysis based on the one or more parameters, wherein the statistical analysis includes one or more of mean deviation analysis, standard deviation analysis, estimation analysis, forecasting analysis, correlation of the one or more parameters, modeling of one or more relationships among the one or more parameters, regression analysis, time series analysis, autoregressive modeling, integrated modeling, moving average modeling, data mining, or probability analysis.

32. An apparatus, comprising:
one or more processing units; and
a memory for storing instructions which, when executed by the one or more processing units, cause the one or more processing units to:
monitor analyte levels by an analyte monitor over a predetermined period of time in which the insulin pump is configured to administer insulin according to a first rate specified by an insulin therapy profile, wherein the analyte monitor comprises a subcutaneous analyte sensor and a transmitter;
retrieve the insulin therapy profile and a predetermined parameter, wherein the predetermined parameter comprises insulin delivery information during the predetermined period of time;
determine a modification based on the insulin therapy profile and at least one of the retrieved insulin delivery information and the monitored analyte levels; and
adjust the insulin therapy profile based on the determined modification to generate an adjusted insulin therapy profile, wherein the adjusted insulin delivery therapy comprises a second rate for administering the insulin.

33. The apparatus of claim 32, wherein the insulin therapy profile comprises at least one of a current basal profile, a temporary basal profile, or a pre-programmed insulin delivery profile.

34. The apparatus of claim 11, wherein the one or more processing units further:
output the adjusted insulin therapy profile to a user device; and
receive, from the user device, an instruction to process the adjusted insulin therapy profile

35. The apparatus of claim 34, wherein the instruction to process the adjusted insulin therapy profile comprises at least one of selecting the adjusted insulin therapy profile, storing the adjusted insulin therapy profile, or ignoring the adjusted insulin therapy profile.

36. The apparatus of claim 35, wherein when the instruction to process the adjusted insulin therapy profile comprises selecting the adjusted insulin therapy profile, the instruction is configured to cause the insulin pump to operate based on the second rate for administering the insulin.

37. The apparatus of claim 32, wherein the second rate for administering the insulin comprises a temporary basal rate.

38. The apparatus of claim 32, wherein the adjusted insulin therapy profile further comprises a new bolus amount determined based on a user input parameter.

39. The apparatus of claim 38, wherein the user input parameter comprises at least one of an amount of carbohydrate to ingest, a type of exercise to perform, and a current time of day information.

40. A method, comprising:
monitoring analyte levels by an analyte monitor over a predetermined period of time in which the insulin pump is configured to administer insulin according to a first rate specified by an insulin therapy profile, wherein the analyte monitor comprises a subcutaneous analyte sensor and a transmitter;
retrieving the insulin therapy profile and a predetermined parameter, wherein the predetermined parameter comprises insulin delivery information during the predetermined period of time;
determining a modification based on the insulin therapy profile and at least one of the retrieved insulin delivery information and the monitored analyte levels; and
adjusting the insulin therapy profile based on the determined modification to generate an adjusted insulin therapy profile, wherein the adjusted insulin delivery therapy comprises a second rate for administering the insulin.
